(19) United States
(12) Patent Application Publication
(10) Pub. No.: US 2013/0108717 A1
(43) Pub. Date: May 2, 2013

(54) COMPOSITIONS FOR THE TREATMENT OF GYNAECOLOGICAL DISORDERS

(71) Applicant: Stefano Togni, Milano (IT)

(72) Inventor: Stefano Togni, Milano (IT)

(73) Assignee: INDENA S.P.A., Milano (IT)

(21) Appl. No.: 13/698,246

(22) PCT Filed: May 3, 2011
(86) PCT No.: PCT/EP11/57052

§ 371 Date: Dec. 14, 2012

(30) Foreign Application Priority Data
May 18, 2010 (IT) MI2010A000884

(51) Int. Cl. A61K 36/758; A61K 31/19; A61K 36/185

(52) U.S. Cl. CPC A61K 36/758; A61K 36/185; A61K 31/19
USPC 424/725

(57) ABSTRACT

The present invention relates to a combination of a rhatany extract, 18β-glycyrrhetic acid, in free form and in complexes with phospholipids, and Zanthoxylum bungeanum extract, for the topical treatment of gynaecological disorders, especially vulvovaginal infections.

### SUMMARY OF THE INVENTION

The present invention relates to a combination of a rhatany extract, 18β-glycyrrhetic acid, in free form or in complexes with phospholipids, and Zanthoxylum bungeanum extract, for the topical treatment of gynaecological disorders, especially vulvovaginal infections.

### INTRODUCTION

Bacterial vaginosis is one of the most frequent forms of vaginal infection. It is due to an alteration of the vaginal flora and its pH, which leads to a reduction in the normal saprophytic flora, especially lactobacilli (Döderlein's bacillus), and abnormal growth of commensal germs like Gardnerella vaginalis, Mycoplasma hominis and anaerobic bacteria (Mobiluncus, Peptostreptococcus, Bacterioides and Eubacterium).

Bacterial vaginosis affects sexually active and pregnant women more frequently than others, and is manifested by a creamy or foamy whitish vaginal discharge, which is foul-smelling (a smell of rotten fish is typical) due to the presence of amines deriving from bacterial metabolism, and an elevated vaginal pH, which instead of being slightly acid presents altered values, greater than 4.5. Microscopic examination shows the classic “clue cells”, namely cells surrounded by bacteria.

Even without causing unpleasant symptoms, bacterial vaginosis can involve gynaecological problems (purulent cervicitis, endometritis, pelvic inflammatory disease and sterility) and psychological problems (repercussions on the sex life due to the bad smell).

Moreover, in obstetric terms, vaginosis can lead to miscarriage, and increase the risk of premature labor due to amniochorial infections, and post-partum endometritis.

Vaginitis is an acute or chronic inflammation of the vagina; if the inflammation also extends to the vulva, it is called vulvovaginitis. The main symptoms, which may be more or less intense, are mainly irritative: stinging, vulvar or vaginal itching, liquid or semiliquid discharge.

The etiological agent is constituted by different species of micro-organisms, the most common being fungi like Candida albicans (candidiasis), Gram (−) bacteria like Gardnerella vaginalis and Staphylococcus aureus (vaginosis), and protozoa like Trichomonas vaginalis (trichomoniasis). Other infectious agents are Neisseria gonorrhoeae, Bacterium coli, and herpes simplex.

Vaginitis is a very common condition: it is estimated that 75 women out of 100 have suffered at least once from vulvovaginitis caused by a fungal infection.

A frequent cause of vaginitis is impoverishment of the saprophytic vaginal bacterial flora (for example after antibiotic treatments) which leads to the onset of opportunistic infections.

The treatment of bacterial vaginosis and vaginitis is generally based on treatments with antibiotics, agents that restore the balance of the vaginal bacterial flora, or a combination of the two.

Treatment with antibiotics (generally local clindamycin, or local or systemic metronidazole) is mainly recommended for pregnant women, symptomatic patients and women due to undergo surgery, because although antibiotics have a rapid effect, their use can worsen the balance of the vaginal bacterial flora by reducing its proliferation, leading to frequent flare-ups.

Probiotic agents restore the balance of the vaginal bacterial flora, stimulating the proliferation of lactobacilli, and thus inhibit the growth of pathogenic bacteria. Preparations based on lactobacilli, and compounds with an acidifying effect on the vaginal pH, generally based on lactic acid or vitamin C, are used for this purpose. However, the use of these agents does not resolve the problem, firstly because the altered vaginal pH can reach such levels of basicity as to prevent the survival of the lactobacilli, and secondly because the effect of the acidifying agents is generally short-lived.

Unfortunately, antibiotic treatment is not always effective in the case of vaginosis and vaginitis, because the increasingly widespread and sometimes indiscriminate use of antibiotics has led, with time, to the development of resistance by the bacteria attacked by these drugs.

Bacteria can become insensitive to antibiotics, deploying various mechanisms, such as:

a) modification of the target of the antibiotic's action

b) production of deactivating enzymes (such as beta-lactamase)

c) impermeabilization of the outer coating of the bacterial cell

d) active outflow systems which expel the antibiotic from the bacterial cell even before the medicament has been able to perform its antibacterial action.

The resistance of various bacterial species to antibiotics tends to increase inexorably year after year, and little research has been conducted into new molecules with antibacterial activity in recent years.

Research is therefore focusing on substances with a different action mechanism from all the other antibiotics, which may be useful to combat pathogenic micro-organisms.

The scientific literature has always taken an interest in the role played by plant derivatives in medical treatment.

On the basis of a blind screening, rhatany (Krameria trianda Ruiz) was recently found to have a particularly interesting role. Its roots and their derivatives have been used by Peruvian populations since ancient times to treat inflammations and lesions of the oral cavity. The medicinal use of this plant was successfully introduced into Europe about two centuries ago.

The plant is currently listed in various pharmacopoeias, which recommend its use in the treatment of inflammatory and infectious disorders affecting the oral cavity, pharynx, tonsils and skin.

The antibacterial and antifungal role of the lipophilic extract of rhatany is due to the presence of particular neolignans and nor-neolignans with a benzofuran structure, in which the lipophilic extract of rhatany is standardized, and which effectively inhibit the proliferation of many micro-organisms, especially Gram-positive bacteria and fungi.

18β-Glycyrrhetic acid is a pentacyclic triterpene, obtained by extraction and hydrolysis from liquorice root (Glycyrrhiza glabra), which possesses significant anti-inflammatory activity.

The complex of 18β-glycyrrhetic acid with phospholipids is described in EP 0283713, and in the present application the complex is indicated by the term “Phytosome®”. When administered topically, the Phytosome® of glycyrrhetic acid also acts as a useful anti-inflammatory, inhibiting oedema of the paw of laboratory animals induced by inoculation of Croton oil, with greater efficacy (approx. 90%) than that of common non-steroidal anti-inflammatory drugs (such as indometacin).

Its activity seems to be associated with inhibition of tissue 11-beta-hydroxysteroid dehydrogenase (11-beta-HSD), which converts cortisol from active to inactive form: by means of this enzymatic interaction, glycyrrhetic acid prolongs the normal anti-inflammatory activity of cortisol, which is released in the tissue site following an inflammatory stimulus. 18β-glycyrrhetic acid Phytosome® can therefore be considered an effective topical

anti-inflammatory of natural origin whose activity corresponds to 25% of that demonstrated by dexamethasone and hydrocortisone, molarity being equal.

Zanthoxylum bungeanum pericarp extract possesses anti-inflammatory and analgesic activity, and can be used to treat itching when applied percutaneously.

### DESCRIPTION OF THE INVENTION

The present invention relates to compositions containing:

a) rhatany extract,

b) 18β-glycyrrhetic acid, either free or in the form of a complex with phospholipids,

and

c) Zanthoxylum bungeanum extract,

for the treatment of gynaecological disorders, especially vaginosis and vaginitis.

It has been found that the compositions according to the invention exert a broad-spectrum antibacterial action against Gram-positive (including methicillin-resistant) micro-organisms, Gram-negative micro-organisms and Candida, together with an anti-inflammatory action, thus providing a useful weapon in the treatment of gynaecological disorders like vaginosis and vaginitis. The antifungal and antibacterial activity exercised by the compositions according to the invention is particularly important, because Candida, which is mainly responsible for gynaecological fungal infections, is notoriously resistant to all the antibiotics currently in use, while methicillin-resistant staphylococci are insensitive to the majority of antibiotics used in clinical practice. The surprising activity of the compositions according to the invention, deriving from the synergic action between the active ingredients towards these micro-organisms, therefore provides doctors with an effective weapon in the topical treatment of vaginosis and vaginitis, which is able to eradicate the pathogens most frequently responsible for these infections. It has been found that the Zanthoxylum bungeanum extract assists in eliminating itching and/or pain.

According to the present invention, the compositions will contain the active ingredients within the following intervals:

a) rhatany extract: 0.01 to 1%, and

b) 18β-glycyrrhetic acid, either free or in the form of a complex with phospholipids: 0.01 to 1%, and

c) Zanthoxylum bungeanum extract: 0.01 to 1%

According to a preferred aspect of the invention, the Zanthoxylum bungeanum extract is prepared as described in EP 1096944.

The pharmacological experiments on the compositions according to the invention are set out below.

### EXPERIMENTAL SECTION

### Activity In Vitro

Materials and Methods

23 microbial strains isolated from the vaginas of patients with different infections were examined.

The strains were the following:
Gardnerella vaginalis (10 vaginally isolated strains)
Candida albicans (5 vaginally isolated strains)
Methicillin-resistant Staphylococcus aureus (5 vaginally isolated strains)
Trichomonas vaginalis (4 vaginally isolated strains)

All the microbial strains were reisolated on specific culture media, namely:
Brucella broth for G. vaginalis
Mannitol salt agar for staphylococci
Sabouraud agar for the Candida strains
Diamond for the Trichomonas vaginalis strains

After seeding, all the media were left to incubate in a thermostat at 37° C. for 24 hours except for the Sabouraud agar, which was incubated at 35° C. for 48 hours.

The substances tested were placed in solution before the tests were conducted, as follows:
rhatany, 15% dried extract, in DMSO
18β-glycyrrhetic acid in 95% ethyl alcohol
Zanthoxylum bungeanum extract, in DMSO

The in vitro antimicrobial activity of the composition according to the invention was evaluated by determining the minimal inhibitory concentrations (MIC), and then compared with that of the individual constituents.

The MICs of the methicillin-resistant S. aureus strains were determined by the method that refers to CLSI document M 7-A5, using the technique of micro-dilution in culture broth. Two-fold serial dilutions of the substances in Mueller-Hinton broth were performed to obtain concentrations of between 1333 and 0.65 mcg/ml. The inoculum was represented by 50.000 CFU/ml. The MIC was defined as the lowest concentration of substance able to prevent bacterial multiplication visible to the naked eye, based on the absence of turbidity of the culture broth.

The method used to determine the MICs of the Gardnerella vaginalis strains refers to CLSI document M11-A4 (broth dilution method). In this case, the medium used for the sensitivity test was Brucella broth with 2.5% of laked horse blood, with the addition of 10% of vitamin complex. After the inoculum, the plates with the wells were incubated in CO2 at 37° C. for 18/24 hours.

The MICs of the Candida strains were determined in accordance with the method that refers to CLSI document M27-A (microdilution in broth method). The medium used to dilute the substances was RPMI 1640 broth with 2% glucose, buffered to pH 7 with 0.165 M MOPS (morpholinepropane-sulphonic acid). The inoculum was prepared in sterile saline solution from strains cultured in Sabouraud agar.

The final inoculum was 2500 CFU/ml. After inoculation, the plates were incubated at 35° C. for 24-48 hours. The MIC was again defined as the lowest concentration of substance visibly able to inhibit growth.

For the Trichomonas vaginalis strains, the MICs were determined on Diamond's medium in triplicate in 96-well plates, with an inoculum of 20000 protozoa per well. The various substances to be tested were added, starting with a concentration of 500 mcg/ml, and followed by two-fold serial dilutions.

The 96-well plates were left to incubate overnight at 37° C. and observed under the optical microscope to evaluate their viability, which was also confirmed with the use of vital stains.

The MIC for the Trichomonas vaginalis strains was defined as the highest dilution of the individual substances at which the protozoa present appeared to lack motility under the optical microscope.

The results of the combination were evaluated on the basis of the fractional inhibitory concentration (FIC) and the FIC index.

The FIC expresses the ratio between the MIC of the combination and that of the substance used alone, while the FIC index identifies the interaction between the two substances, and is the sum of the FICs of the individual substances.

The FIC and the FIC index were calculated as follows:

$$FIC = \frac{MIC of the combination A + B + C}{MIC of substance A or B or C alone}$$

FIC index=FIC of substance A+FIC of substance B+FIC of substance C.

The combination of two substances is considered synergic if the FIC index is equal to or less than 0.5, indifferent if it is greater than 0.5 and less than 2, and antagonistic if the value is greater than 2.

Results

The results of the tests set out in the Table show a surprising synergic activity of the ingredients of the composition. The results on the Candida albicans strains are particularly important, as the combination presents a surprising activity, greater than that of rhatany extract, the other ingredients being substantially inert.

In view of the in vitro activity of rhatany and 18β-glycyrretic acid, the zanthoxylum extract being substantially inert, on the strains responsible for vaginal infection, and with a view to the topical use of these substances, it will be possible to reach concentrations far higher than the MIC at the site of the infection, providing an excellent ability to eradicate the pathogens which appear sensitive or moderately sensitive to these substances in vitro.

TABLE
Antimicrobial activity of the compositions according to the
invention. MIC expressed in mcg/ml.

| | Dried | | | | |
|---|---|---|---|---|---|
| | rhatany | 18-beta- | Zanthoxylum | | |
| | extract | glycyrrhetic | bungeanum | | FIC |
| STRAINS | 0.2% | acid 0.375% | extract 0.5% | Combination | ind. |
| Gardnerella | 333 | 667 | >1333 | 83 | 0.43 |
| vaginalis 1 | | | | | |
| Gardnerella | 667 | 667 | >1333 | 167 | 0.56 |
| vaginalis 2 | | | | | |
| Gardnerella | 333 | 333 | >1333 | 50 | 0.33 |
| vaginalis 3 | | | | | |
| Gardnerella | 333 | 167 | >1333 | 50 | 0.51 |
| vaginalis 4 | | | | | |
| Gardnerella | 667 | 667 | >1333 | 83 | 0.3 |
| vaginalis 5 | | | | | |
| Gardnerella | 667 | 667 | >1333 | 83 | 0.3 |
| vaginalis 6 | | | | | |
| Gardnerella | 333 | 167 | >1333 | 50 | 0.48 |
| vaginalis 7 | | | | | |
| Gardnerella | 333 | 333 | >667 | 50 | 0.38 |
| vaginalis 8 | | | | | |
| Gardnerella | 333 | 333 | >667 | 50 | 0.38 |
| vaginalis 9 | | | | | |
| Gardnerella | 333 | 167 | >1333 | 50 | 0.48 |
| vaginalis 10 | | | | | |
| Trichomonas | 150 | 150 | >667 | 25 | 0.36 |
| vaginalis 1 | | | | | |
| Trichomonas | 150 | 150 | >1333 | 25 | 0.35 |
| vaginalis 2 | | | | | |
| Trichomonas | 150 | 150 | >1333 | 25 | 0.35 |
| vaginalis 3 | | | | | |
| Trichomonas | 300 | 150 | >667 | 25 | 0.25 |
| vaginalis 4 | | | | | |
| Candida | 84 | 667 | >1333 | 22 | 0.32 |
| albicans 1 | | | | | |
| Candida | 42 | 667 | >1333 | 11 | 0.31 |
| albicans 2 | | | | | |
| Candida | 42 | 667 | >1333 | 11 | 0.31 |
| albicans 3 | | | | | |
| Candida | 84 | 667 | >1333 | 11 | 0.31 |
| albicans 4 | | | | | |
| Candida | 84 | 333 | >667 | 22 | 0.33 |
| albicans 5 | | | | | |
| S. aureus Met | 42 | >1333 | 1333 | 11 | 0.28 |
| R 1 | | | | | |
| S. aureus Met | 84 | >1333 | >667 | 22 | 0.3 |
| R 2 | | | | | |
| S. aureus Met | 42 | 1333 | >1333 | 11 | 0.28 |
| R 3 | | | | | |
| S. aureus Met | 42 | 1333 | 1333 | 11 | 0.28 |
| R 4 | | | | | |
| S. aureus Met | 42 | >1333 | >667 | 11 | 0.29 |
| R 5 | | | | | |

In Vivo Activity

Materials and Methods

The cream described in Formulation Example 2 was applied once a day for two weeks with a disposable dispenser in 22 women (age range 25-50 years) with a vaginal pH>6.5, after they had given their informed consent.

At baseline and at the end of treatment, instrumental evaluations such as the following were performed: measurement of pH and vaginal humidity, and a vaginal swab to determine the bacterial and fungal count. The women were asked to lead a normal life with their usual sexual habits, apart from sexual intercourse, which was not to take place in the two days prior to the swab.

Results

17 women completed the trial, with the following results:

the pH significantly declined (p<0.01), from a mean value of 7.89 to a value of 5.01, indicating regularization of the physiological vaginal conditions.

The bacteria count fell significantly, from 3.52×10⁷ to 9.95×10⁶ after treatment (−72%). The fungal count also fell significantly, by 32%, from 1.48×10⁴ to 1.01×10⁴.

47% of the women reported an improvement in the previous condition of vaginal dryness, and 38% reported a reduction in the previous stinging/smarting sensation.

The formulations according to the invention can be prepared according to well-known conventional methods, such as those described in “Remington's Pharmaceutical Handbook”, Mack Publishing Co., N.Y., USA, together with suitable excipients.

The compositions according to the invention will be conveniently formulated in water/oil emulsions and other compatible excipients for treatment of the genital mucosae; for internal treatments the compounds will be formulated as pessaries which disintegrate readily after introduction into the vaginal meatus.

Examples of formulations according to the invention include creams, ointments, lotions, pessaries or equivalent formulations, including capsules that dissolve at internal body temperature.

Some examples of formulations according to the invention are set out below.

### Formulation Example 1

### Vaginal Douche

| Ingredient (INCI) | %: |
|---|---|
| Water | 81.11750 |
| Caprylic/capryl glucoside | 3.00000 |
| Sodium lauroyl sarcosinate | 2.40000 |
| Sodium cocoyl wheat amino acids | 1.50000 |
| Peg-18 glyceryl oleate/cocoate | 1.50000 |
| Cocoyl proline | 1.40000 |
| Aesculus hippocastanum extract | 1.00000 |
| Sodium lauroyl oat amino acids | 0.60000 |
| Phenoxyethanol | 0.50000 |
| Lactic acid | 0.36000 |
| Potassium undecylenoyl hydrolyzed soy protein | 0.30000 |
| Imidazolidinyl urea | 0.30000 |
| Oleyl alcohol | 0.16000 |
| Lavandula angustifolia oil | 0.10000 |
| Propylene glycol | 0.09800 |
| Hydrolyzed vegetable protein | 0.08750 |
| Krameria triandra extract | 0.05000 |
| Zanthoxylum bungeanum extract | 0.04000 |
| 18β-Glycyrrhetic acid | 0.07500 |

### Formulation Example 2

### Vaginal Cream

INGREDIENTS

| (common name) | % w/w |
|---|---|
| Water | 55.000 |
| Sodium benzoate | 0.500 |
| D-Panthenol | 0.500 |
| Glycyrrhetic acid phytosome | 0.125 |
| Xanthan gum | 0.300 |
| Veegum Ultra | 2.000 |
| Brij 72 | 6.000 |
| Brij 721 | 3.000 |
| Lanette 16 | 2.000 |
| White wax | 0.500 |
| 18β-Glycyrrhetic acid | 0.375 |
| Vitamin E acetate | 0.500 |
| Silkflo 364 | 6.000 |
| Cosmacol ELI | 5.000 |
| Titanium dioxide | 3.000 |
| Optiphen | 1.500 |
| Vegetable glycerin FU | 5.000 |
| Propylene glycol USP | 7.000 |
| Dried rhatany extr. | 0.200 |
| Zanthoxylum bungeanum extract | 0.500 |
| Simulgan INS 100 | 1.000 |

### Formulation Example 3

### Vaginal Pessary

INGREDIENTS

| (common name) | % w/w | |
|---|---|---|
| 18β-Glycyrrhetic acid complex with phospholipids | 0.5 | g |
| Dried rhatany extract | 0.2 | g |
| Zanthoxylum bungeanum extract | 0.05 | g |
| Glycerides of fatty acids q.s for | 20 | g |

1. Compositions comprising:
a) rhatany extract,
b) 18β-glycyrrhetic acid, either free or in the form of a complex with phospholipids, and
c) Zanthoxylum bungeanum extract.

2. Compositions according to claim 1, wherein the active ingredients are present within the following ranges (by weight per unit dose):
a) rhatany extract: 0.01 to 1%,
b) 18β-glycyrrhetic acid, either free or in the form of a complex with phospholipids: 0.01 to 1%, and
c) Zanthoxylum bungeanum extract: 0.01 to 1%.

3. Compositions according to claim 1, wherein the composition is formulated in the form of oil/water emulsions, soft gelatin capsules, vaginal pessaries or equivalent formulations, creams, ointments or lotions.

4. A method for treating vaginal disorders comprising the compositions of claim 1 comprising:
a) rhatany extract,
b) 18β-glycyrrhetic acid, either free or in the form of a complex with phospholipids, and
c) Zanthoxylum bungeanum extract.

5. The method according to claim 4, wherein the vaginal disorders are vaginosis and vaginitis.
